SEQUENCE LISTING (4 sequences)

SEQ ID NO: 1 - protein, 270 aa, Artificial Sequence
  variable domain of CAR sequence
METDTLLLWVLLLWVPGSTGDIVLTQSPASLAVSLGQRATMSCRAGESVDIFGVGFLHWYQQKPGQPPKLLIYRASNLESGIPVRFSGTGSRTDFTLIIDPVEADDVATYYCQQTNEDPYTFGGGTKLEIKGSTSGSGKPGSGEGSTKGEVQLQQSGAELVEPGASVKLSCTASGFNIKDTYMHWVKQRPEQGLEWIGRIDPANGNSKYVPKFQGKATITADTSSNTAYLQLTSLTSEDTAVYYCAPFGYYVSDYAMAYWGQGTSVTVSS

SEQ ID NO: 2 - protein, 46 aa, Artificial Sequence
  hinge domain of CAR sequence
AKPTTTPAPRPPTPAPTIASQPLSLRPEACRPAAGGAVHTRGLDFA

SEQ ID NO: 3 - protein, 138 aa, Artificial Sequence
  effector domain of CAR sequence
LDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 4 - protein, 462 aa, Artificial Sequence
  CAR sequence
METDTLLLWVLLLWVPGSTGDIVLTQSPASLAVSLGQRATMSCRAGESVDIFGVGFLHWYQQKPGQPPKLLIYRASNLESGIPVRFSGTGSRTDFTLIIDPVEADDVATYYCQQTNEDPYTFGGGTKLEIKGSTSGSGKPGSGEGSTKGEVQLQQSGAELVEPGASVKLSCTASGFNIKDTYMHWVKQRPEQGLEWIGRIDPANGNSKYVPKFQGKATITADTSSNTAYLQLTSLTSEDTAVYYCAPFGYYVSDYAMAYWGQGTSVTVSSPLEPAKPTTTPAPRPPTPAPTIASQPLSLRPEACRPAAGGAVHTRGLDFAPEFRLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2020/0163995 A1
(43) Pub. Date: May 28, 2020

(54) PHARMACEUTICAL KIT AND USES THEREOF

(71) Applicant: Johnpro Biotech Inc., Taipei City (TW)

(72) Inventors: Kwan-Hwa CHI, Taipei City (TW); Yu-Shan WANG, Taipei City (TW); Yi-Chun HUANG, Taipei City (TW); Hsin-Chien CHIANG, Taipei City (TW)

(73) Assignee: Johnpro Biotech Inc., Taipei City (TW)

(21) Appl. No.: 16/636,907

(22) PCT Filed: Jul. 23, 2018
(86) PCT No.: PCT/CN2018/096703

§ 371 Date: Feb. 6, 2020

Related U.S. Application Data
(60) Provisional application No. 62/541,778, filed on Aug. 6, 2017.

(51) Int. Cl. A61K 35/17; A61K 38/17; A61K 31/706; A61K 31/165; A61K 31/201; A61K 31/19; A61P 35/00

(52) U.S. Cl. CPC A61K 35/17; A61K 38/1774; A61K 31/706; A61P 35/00; A61K 31/201; A61K 31/19; A61K 31/165

(57) ABSTRACT

Disclosed herein is a pharmaceutical kit for treating cancers. The present pharmaceutical kit comprises an agent and an engineered natural killer cell. The agent is capable of increasing a tumor-associated antigen expression in cancer cells, which can then be targeted and destroyed by the engineered natural killer cell having a tumor-associated antigen-specific chimeric antigen receptor. Also disclosure herein is the uses of the present pharmaceutical kit for the treatment of cancers.

### CROSS-REFERENCE TO RELATED APPLICATIONS

This application relates to and claims the benefit of U.S. Provisional Application No. 62/541,778, filed Aug. 6, 2017; the content of the application is incorporated herein by reference in its entirety.

### BACKGROUND OF THE INVENTION

### 1. Field of the Invention

The present disclosure in general relates to the field of cancer treatment. More particularly, the present disclosure relates to a pharmaceutical kit and the uses thereof for preparation of a medicament for treating cancers.

### 2. Description of Related Art

Cancer is a complex disease characterized by the abnormal and unregulated growth of cells. Cancer cells are different from normal cells in many ways, including (1) cell communication: compared with normal cells, cancer cells are less responsive to signals that regulate the growth or death of cells; (2) invasive ability: cell adhesion molecules are usually down-regulated in cancer cells; accordingly, the less restricted cells may easily metastasize or spread to other areas of the body via blood or lymph fluid; (3) cell specialization: cancer cells are unspecialized or less differentiated as compared to normal cells; and (4) immunosuppression: cancer cells suppress immune response via activating various immunosuppressive cells (e.g., regulatory T cells (Tregs) or myeloid-derived suppressor cells (MDSCs)) and/or stimulating the expression of immunosuppressive factors (e.g., vascular endothelial growth factor (VEGF), transforming growth factor-beta (TGF-β) or interleukin-10 (IL-10)).

The most common treatments for cancers include surgery, radiation therapy and chemotherapy. Unfortunately, in addition to suppressing tumor growth, these treatments are also associated with risks of injury or cytotoxicity to normal tissues. Accordingly, alternative treatments designed to kill cancer cells without producing the side-effects are being tried in research and pre-clinical studies. Among these treatments, immunotherapy is one of the most promising treatment that eliminates tumors via activating tumor-specific immune cells (e.g., T cells, B cells, dendritic cells (DCs), natural killer cells (NK cells) and natural killer T cells (NKT cells)), and/or stimulating the expression/release of anti-cancer factors (e.g., interferon-γ (IFN-γ) and granzymes). The activated immune cells are characterized by their targeting specificity; that is, these immune cells can specifically target to cancer cells via recognizing and binding to the tumor-associated antigen (TAA) overexpressed or uniquely expressed on cancer cells. Nevertheless, the therapeutic efficacy of immunotherapy in clinical practice is still disappointed due to the fact that both the major histocompatibility complex (MHC) and the TAA presented thereon are often down-regulated or lost on cancer cells, one of mechanisms for escaping immune surveillance. Besides, it is reported that the therapeutic efficacy of NK cells may be compromised by immunosuppressive factors (e.g., TGF-β or IL-10) secreted by cancer cells.

In view of the foregoing, there exists in the related art a need for an improved method for efficiently treating a cancer patient, and accordingly, improving the life quality and/or lifespan of the cancer patient.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, one aspect of the disclosure is directed to a pharmaceutical kit useful in treating a subject having or suspected of having a cancer. The present pharmaceutical kit comprises a first container containing an agent, and a second container containing an engineered natural killer (NK) cell. According to embodiments of the present disclosure, the agent is capable of increasing the expression of a tumor-associated antigen (TAA) on the cancer, and the engineered NK cell has a chimeric antigen receptor (CAR) specific to the TAA.

Another aspect of the present disclosure pertains to a method of treating a subject having or suspected of having a cancer by use of the present pharmaceutical kit. The method comprises administering to the subject a first effective amount of the present agent to increase the expression of a TAA on the cancer; and administering to the subject a second effective amount of the present engineered NK cell having a CAR specific to the TAA.

According to some embodiments of the present disclosure, the TAA is carcinoembryonic antigen (CEA). In these embodiments, the variable domain, the hinge domain and the effector domain of the CAR respectively comprise the amino acid sequences at least 85% identical to SEQ ID NOs: 1, 2 and 3. According to the working example, the CAR comprises the amino acid sequence at least 85% identical to SEQ ID NO: 4.

In general, the agent is selected from the group consisting of, 5-azacytidine, 5,6-dihydro-5-azacytidine, 5-aza-2′-deoxycytidine, arabinofuranosyl-5-azacytosine, trichostatin A (TSA), phenylbutyrate (PB), sodium butyrate (NaB), valproic acid (VPA), and suberoylanilide hydroxamic acid (SAHA). According to one working example, the agent is 5-azacytidine or sodium butyrate.

Exemplary cancers treatable with the present pharmaceutical kit and/or method include, but are not limited to, gastric cancer, lung cancer, bladder cancer, breast cancer, pancreatic cancer, renal cancer, colon cancer, rectal cancer, cervical cancer, ovarian cancer, brain tumor, prostate cancer, hepatocellular carcinoma, melanoma, esophageal carcinoma, multiple myeloma, and head and neck squamous cell carcinoma. According to some embodiments of the present disclosure, the cancer is resistant to chemotherapy, radiation therapy or immunotherapy.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings, where:

FIG. 1 is a histogram that depicts the CEA expression in specified cancer cells according to Example 2 of the present disclosure.

FIG. 2 is a line chart that depicts the cytotoxic effect of NK92MI-CEA cells on specified cancer cells according to Example 2 of the present disclosure.

FIGS. 3A and 3B are line charts respectively depicting the cytotoxic effect of NK92MI-CEA cells on 5-azacytidine treated cancer cells (FIG. 3A) and on sodium butyrate treated cancer cells (FIG. 3B) according to Example 3 of the present disclosure.

FIGS. 4A-4C are line chart and histograms respectively depicting the tumor volume (FIGS. 4A and 4B) and the CEA serum level (FIG. 4C) of mice administered with specified treatments according to Example 4 of the present disclosure.

### DETAILED DESCRIPTION OF THE INVENTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### 1. DEFINITIONS

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

“Percentage (%) amino acid sequence identity” with respect to the polypeptide sequences identified herein is defined as the percentage of polypeptide residues in a candidate sequence that are identical with the amino acid residues in the specific polypeptide sequence, after aligning the sequences and introducing gaps, if necessary, to achieve the maximum percent sequence identity, and not considering any conservative substitutions as part of the sequence identity. Alignment for purposes of determining percentage sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, sequence comparison between two polypeptide sequences was carried out by computer program Blastp (protein-protein BLAST) provided online by Nation Center for Biotechnology Information (NCBI). The percentage amino acid sequence identity of a given polypeptide sequence A to a given polypeptide sequence B (which can alternatively be phrased as a given polypeptide sequence A that has a certain % amino acid sequence identity to a given polypeptide sequence B) is calculated by the formula as follows:

$$\frac{X}{Y} \times 100 \%$$

where X is the number of amino acid residues scored as identical matches by the sequence alignment program BLAST in that program's alignment of A and B, and where Y is the total number of amino acid residues in A or B, whichever is shorter.

As used herein, the term “cycle”, “cycle of treatment” and “treatment cycle” are interchangeable and refer to a period of time, during which the treatment is administered to the patient. Typically, in cancer therapy a cycle of treatment is followed by a rest period during which no treatment is given. Following the rest period, one or more further cycles of treatment may be administered, each followed by additional rest periods.

The term “treating” encompasses partially or completely preventing, ameliorating, mitigating and/or managing a symptom, a secondary disorder or a condition associated with cancers. The term “treating” as used herein refers to application or administration of one or more compounds/cells of the present disclosure to a subject, who has a symptom, a secondary disorder or a condition associated with cancers, with the purpose to partially or completely alleviate, ameliorate, relieve, delay onset of, inhibit progression of, reduce severity of, and/or reduce incidence of one or more symptoms, secondary disorders or features associated with cancers. Symptoms, secondary disorders, and/or conditions associated with cancers include, but are not limited to, fever, weakness, fatigue, weight loss, pain, cough, bleeding, skin change, diarrhea or constipation, nausea, vomiting, and loss of appetite. Treatment may be administered to a subject who exhibits only early signs of such symptoms, disorder, and/or condition for the purpose of decreasing the risk of developing the symptoms, secondary disorders, and/or conditions associated with cancers. Treatment is generally “effective” if one or more symptoms or clinical markers are reduced as that term is defined herein. Alternatively, a treatment is “effective” if the progression of a symptom, disorder or condition is reduced or halted.

The term “effective amount” as referred to herein designate the quantity of a component which is sufficient to yield a desired response. For therapeutic purposes, the effective amount is also one in which any toxic or detrimental effects of the component are outweighed by the therapeutically beneficial effects. The specific effective or sufficient amount will vary with such factors as the particular condition being treated, the physical condition of the patient (e.g., the patient's body mass, age, or gender), the type of mammal or animal being treated, the duration of the treatment, the nature of concurrent therapy (if any), and the specific formulations employed and the structure of the compounds or its derivatives. Effective amount may be expressed, for example, in cell number, grams, milligrams or micrograms or as milligrams per kilogram of body weight (mg/Kg). Alternatively, the effective amount can be expressed in the density of the active component (e.g., the present engineered NK cell), such as cell number per volume of medium; or be expressed in the concentration of the active component (e.g., the present agent), such as molar concentration, mass concentration, volume concentration, molality, mole fraction, mass fraction and mixing ratio. Specifically, the term “therapeutically effective amount” used in connection with the agent or the engineered NK cell described herein refers to the quantity of the agent or the engineered NK cell, which is sufficient to alleviate or ameliorate the symptoms associated with the cancer in the subject. Persons having ordinary skills could calculate the human equivalent dose (HED) for the medicament (such as the present agent) based on the doses determined from animal models. For example, one may follow the guidance for industry published by US Food and Drug Administration (FDA) entitled “Estimating the Maximum Safe Starting Dose in Initial Clinical Trials for Therapeutics in Adult Healthy Volunteers” in estimating a maximum safe dosage for use in human subjects.

As used herein, the term “tumor-associated antigen” or “TAA” includes proteins or polypeptides that are preferentially expressed on the surface of a tumor/cancer cell. The expression “preferentially expressed”, as used in this context, means that the antigen is expressed on a tumor cell at a level that is at least 10% greater (e.g., 10%, 20%, 30%, 40%, 50%, 60%, 70%, 80%, 90%, 100%, 110%, 150%, 200%, 400%, or more) than the expression level of the antigen on non-tumor cells. In certain embodiments, the antigen is an antigen that is preferentially expressed on the surface of a tumor cell selected from the group consisting of, gastric cancer, lung cancer, bladder cancer, breast cancer, pancreatic cancer, renal cancer, colon cancer, rectal cancer, cervical cancer, ovarian cancer, brain tumor, prostate cancer, hepatocellular carcinoma, melanoma, esophageal carcinoma, multiple myeloma, and head and neck squamous cell carcinoma.

As used herein, the term “chimeric antigen receptor” or “CAR” refers to an engineered receptor used to confer the specificity of an antibody onto a cell, such as a T cell or a NK cell. More specifically, the engineered receptor comprises an extracellular domain capable of binding to an antigen, a transmembrane domain derived from a polypeptide different from a polypeptide from which the extracellular domain is derived, and at least one intracellular domain. The “chimeric antigen receptor” is sometimes called a “chimeric receptor”, a “T-body”, or a “chimeric immune receptor (CIR).” The “extracellular domain capable of binding to an antigen” means any oligopeptide or polypeptide that can bind to a certain antigen. The “intracellular domain” means any oligopeptide or polypeptide known to function as a domain that transmits a signal to cause activation or inhibition of a biological process in a cell. The “transmembrane domain” means any oligopeptide or polypeptide known to span the cell membrane and that can function to link the extracellular and signaling domains. A chimeric antigen receptor may optionally comprise a “hinge domain” which serves as a linker between the extracellular and transmembrane domains.

The term “engineer,” “engineering” or “engineered,” as used herein, refers to genetic manipulation or modification of biomolecules such as DNA, RNA and/or protein, or like technique commonly known in the biotechnology art.

The term “subject” refers to a mammal including the human species that is treatable with methods of the present invention. The term “subject” is intended to refer to both the male and female gender unless one gender is specifically indicated.

### 2. DETAIL DESCRIPTION OF PREFERRED EMBODIMENTS

The expression/overexpression of TAAs is usually associated with tumorigenesis. For example, some TAAs (e.g., growth factors or the receptors thereof, signal transducers, and transcription factors) are known to induce cellular proliferation or invasion, while other TAAs (e.g., cytokines or the receptors thereof and immune checkpoints) play a role in escaping the immune surveillance. Further, it has been reported that TAA may relates to the resistance of chemotherapy in cancer cells. Based on the tumor-promoting property of TAA, most of the current treatments are developed either to neutralize or down-regulate the TAA expression so as to achieve an anti-tumor effect. The present invention is based at least, in part, on the finding that instead of promoting tumorigenesis, the agents inducing TAA expression may additively or synergistically enhance the anti-tumor effect of immunotherapy.

Accordingly, the first aspect of the present disclosure is directed to a pharmaceutical kit for treating a subject in need thereof, for example, a subject having or suspected of having a cancer. According to embodiments of the present disclosure, the present pharmaceutical kit comprises a first container containing therein a first agent that enhances the expression level of a TAA on the cancer; and a second container containing therein an engineered NK cell, which has a CAR that specifically recognizes and binds to the TAA. Once the present pharmaceutical kit is administered to the subject, the engineered NK cell having the TAA-specific CAR will specifically target and destroy the cancer cell having the TAA expressed thereon.

In general, the agent that enhances the expression level of TAA on the cancer may be a histone deacetylase (HDAC) inhibitor, for example, trichostatin A, phenylbutyrate, sodium butyrate, valproic acid, and suberoylanilide hydroxamic acid. Alternatively, the agent may be a DNA demethylating agent, such as 5-azacytidine, 5,6-dihydro-5-azacytidine, 5-aza-2′-deoxycytidine, and arabinofuranosyl-5-azacytosine. According to one working example of the present disclosure, the agent is 5-azacytidine. According to another working example of the present disclosure, the agent is sodium butyrate.

Optionally, the agent may be a polypeptide having the ability to stimulate or enhance TAA expression (e.g., CEA expression) on the cancer; for example, a recombinant interferon (e.g., recombinant IFN-α, IFN-β and IFN-γ). The polypeptide may be prepared by a method familiar with the skilled artisan; for example, introducing a polynucleotide encoding the polypeptide into a suitable cell (e.g., 293T) so as to express and produce the polypeptide therein. Alternatively, the polypeptide may be synthesized by commonly used methods such as t-BOC or FMOC protection of alpha-amino groups. Both methods involve stepwise syntheses whereby a single amino acid is added at each step starting from the C terminus of the peptide. Polypeptides of the invention can also be synthesized by the well-known solid phase peptide synthesis methods.

Non-limiting examples of TAA include CEA, CD19, CD20, CD23, CD30, CD56, CD73, CD123, alpha-fetoprotein (AFP), cancer antigen 125 (CA-125; also known as mucin 16 or MUC 16), mucin 1 (MUC-1), CO17-1A (also known as GA733, KS1-4, KSA or EpCAM), prostatic specific antigen (PSA), prostate stem cell antigen (PSCA), melanoma-associated antigen (MAA), tyrosinase, elastase, cathepsin G (CatG), Wilms tumor (WT1), fibroblast growth factor 5 (FGF-5), insulin-like growth factor receptor-1 (IGF-1R), Lewis(y) antigen, mutated p53, mutated ras, human epidermal growth factor receptor 2 (HER2; also known as Neu, ErbB-2 or CD340), epidermal growth factor receptor (EGFR), vascular endothelial growth factor receptor 2 (VEGFR2), platelet derived growth factor receptor (PDGFR), folate binding protein (FBP), HIV-1 envelope glycoprotein gp120, HIV-1 envelope glycoprotein gp41, GD2, GD3, c-Met (also known as hepatocyte growth factor receptor or HGF receptor), mesothelin (MSLN), human endogenous retrovirus-K (HERV-K), IL-11R-alpha, survivin, and chondroitin sulfate proteoglycan 4 (CSPG4). According to some embodiments of the present disclosure, the TAA is CEA.

NK cells comprised in the present pharmaceutical kit are preferably engineered to express thereon chimeric receptors that specifically recognize and bind to corresponding TAAs of the cancer cells. The methods useful in engineering NK cells include, but are not limited to, transfection method (i.e., introducing a polynucleotide into NK cells by physical and/or chemical treatment), viral transduction method (i.e., introducing a polynucleotide into NK cells by a virus or a viral vector), and nucleofection (i.e., applying NK cells with a specific voltage and reagent so as to introduce a polypeptide into the NK cells). According to one embodiment of the present disclosure, the present engineered NK cell is produced by lentiviral transduction.

Preferably, each of the present NK cells is engineered to express a CAR specific to CEA, in which the CAR comprises, from N-terminus to C-terminus, a variable domain, a hinge domain and an effector domain. According to some embodiments of the present disclosure, the variable domain useful in recognizing CEA comprises the amino acid sequence at least 85% identical to SEQ ID NO: 1; that is, the CEA-specific variable domain may be 85%, 86%, 87%, 88%, 89%, 90%, 91%, 92%, 93%, 94%, 95%, 96%, 97%, 98%, 99% or 100% identical to SEQ ID NO: 1. Preferably, the amino acid sequence of the CEA-specific variable domain is at least 90% identical to SEQ ID NO: 1. More preferably, the amino acid sequence of the CEA-specific variable domain is at least 95% identical to SEQ ID NO: 1. According to one working example of the present disclosure, the CEA-specific variable domain comprises the amino acid sequence 100% identical to SEQ ID NO: 1.

The hinge domain serves as a linker to link the variable domain and the effector domain. In general, the hinge domain may influence the stability, expression and function of the CAR. According to certain embodiments of the present disclosure, the hinge domain of the present CAR comprises the amino acid sequence at least 85% identical to SEQ ID NO: 2, for example, 85%, 86%, 87%, 88%, 89%, 90%, 91%, 92%, 93%, 94%, 95%, 96%, 97%, 98%, 99% or 100% identical to SEQ ID NO: 2. Preferably, the amino acid sequence of the hinge domain is at least 90% identical to SEQ ID NO: 2. More preferably, the amino acid sequence of the hinge domain is at least 95% identical to SEQ ID NO: 2. According to one specific example of the present disclosure, the hinge domain comprises the amino acid sequence 100% identical to SEQ ID NO: 2.

The effector domain of the present CAR transmits the activation signal to the NK cell that induces the NK cell to destroy the CEA-repressing cancer cells. According to certain embodiments of the present disclosure, the effector domain of the present CAR comprises the amino acid sequence at least 85% identical to SEQ ID NO: 3, for example, 85%, 86%, 87%, 88%, 89%, 90%, 91%, 92%, 93%, 94%, 95%, 96%, 97%, 98%, 99% or 100% identical to SEQ ID NO: 3. Preferably, the amino acid sequence of the effector domain is at least 90% identical to SEQ ID NO: 3. More preferably, the amino acid sequence of the effector domain is at least 95% identical to SEQ ID NO: 3. According to one specific example of the present disclosure, the effector domain comprises the amino acid sequence 100% identical to SEQ ID NO: 3.

According to certain embodiments of the present disclosure, the present CAR comprises the amino acid sequence at least 85% identical to SEQ ID NO: 4, for example, 85%, 86%, 87%, 88%, 89%, 90%, 91%, 92%, 93%, 94%, 95%, 96%, 97%, 98%, 99% or 100% identical to SEQ ID NO: 4. Preferably, the amino acid sequence of the present CAR is at least 90% identical to SEQ ID NO: 4. More preferably, the amino acid sequence of the present CAR is at least 95% identical to SEQ ID NO: 4. According to one working example of the present disclosure, the present CAR comprises the amino acid sequence 100% identical to SEQ ID NO: 4.

The containers suitable for holding the agent and/or the engineered NK cells may be formed from a variety of materials such as glass, or plastic. The first container may hold the present agent or a pharmaceutical formulation thereof, in an amount effective for enhancing TAA expression of a cancer. The second container may hold the present engineered NK cells or a pharmaceutical formulation thereof, in an amount effective for killing the cancer. The kit may further comprise a label or package insert on or associated with the containers. The label or package insert indicates that the agent and engineered NK cells respectively housed in the first and second containers are used for treating specified cancer. Alternatively or additionally, the kit may further comprise a third container comprising a pharmaceutically acceptable buffer, such as a phosphate-buffered saline (PBS), Ringer's solution or dextrose solution. It may further include other materials desirable from a commercial and user standpoint, including other buffers, diluents, filters, needles, and syringes. The kit may further include directions for the administration of the agent and the engineered NK cells.

The second aspect of the present disclosure is directed to a method of treating a subject in need thereof (e.g., a subject suffering from a cancer, or a subject suspected of having a cancer) by use of the present pharmaceutical kit. The method comprises the steps of,

(a) administering to the subject a first effective amount of the present agent; and

(b) administering to the subject a second effective amount of the present engineered NK cell.

In the step (a), the present agent is administered to the subject thereby increasing the TAA expression on cancer cells. According to some embodiments, the subject is a mouse, in which the agent is administered in the amount of 0.1 mg to 1 Kg per Kg of body weight of the subject per day (i.e., 0.1 mg-1 Kg/Kg/day). Preferably, the agent is administered in the amount of 1 mg-100 g/Kg/day. More preferably, the agent is administered in the amount of 10 mg-10 g/Kg/day. According to one working example of the present disclosure, 100-500 mg/Kg/day of the present agent is sufficient to increase the TAA expression on cancer cells thereby enhancing the anti-tumor effect of the present engineered NK cell.

A skilled artisan could calculate the human equivalent dose (HED) for the agent based on the doses determined from animal models. Accordingly, the agent is administered to the human in the amount of 1 g-100 g (1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980 or 990 μg; or 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980 or 990 mg; or 1, 2, 3, 4, 5, 6, 7, 8, 9, 10, 20, 30, 40, 50, 60, 70, 80, 90 or 100 g) per Kg of body weight of the subject per day. Preferably, the agent is administered to the human in the amount of 10 μg-10 g/Kg/day. More preferably, the agent is administered to the human in the amount of 100 μg-1000 mg/Kg/day.

Alternatively, the agent may be administered in accordance with the body surface of the subject. For example, when the subject is a mouse, then the agent may be administered in the amount of 0.1 mg-1 Kg per m² of body surface of the subject per day (0.1 mg-1 Kg/m²/day). In this case, the HED is about 1 μg-100 g/m²/day.

Depending on the desired purpose, the agent may be administered by any suitable route, for example, by enteral, oral, nasal, parenteral (such as intratumoral, intramuscular, intravenous, intraarterial, subcutaneous, intraperitoneal, intracerebral, intracerebroventricular or intrathecal injection), topical or transmucosal administration.

For the purpose of efficiently increasing the TAA expression, the agent may be administered to the subject one or more times. For example, the agent may be administered once for a full course of treatment. Alternatively, the agent may be administered to the subject daily for at least 7 days; for example, 7, 8, 9, 10, 11, 12, 13, 14, 15, 16, 17, 18, 19, 20, 21, 22, 23, 24, 25, 26, 27, 28 or more days. According to certain embodiments of the present disclosure, sodium butyrate is administered to the subject daily for 9 doses so as to enhance TAA expression on cancer cells.

In the step (b), the engineered NK cell is administered to the subject in the amount of 1×10⁴-5×10¹¹ cells (e.g., 1×10⁴, 1.5×10⁴, 2×10⁴, 2.5×10⁴, 3×10⁴, 3.5×10⁴, 4×10⁴, 4.5×10⁴, 5×10⁴, 5.5×10⁴, 6×10⁴, 6.5×10⁴, 7×10⁴, 7.5×10⁴, 8×10⁴, 8.5×10⁴, 9×10⁴, 9.5×10⁴, 1×10⁵, 1.5×10⁵, 2×10⁵, 2.5×10⁵, 3×10⁵, 3.5×10⁵, 4×10⁵, 4.5×10⁵, 5×10⁵, 5.5×10⁵, 6×10⁵, 6.5×10⁵, 7×10⁵, 7.5×10⁵, 8×10⁵, 8.5×10⁵, 9×10⁵, 9.5×10⁵, 1×10⁶, 1.5×10⁶, 2×10⁶, 2.5×10⁶, 3×10⁶, 3.5×10⁶, 4×10⁶, 4.5×10⁶, 5×10⁶, 5.5×10⁶, 6×10⁶, 6.5×10⁶, 7×10⁶, 7.5×10⁶, 8×10⁶, 8.5×10⁶, 9×10⁶, 9.5×10⁶, 1×10⁷, 1.5×10⁷, 2×10⁷, 2.5×10⁷, 3×10⁷, 3.5×10⁷, 4×10⁷, 4.5×10⁷, 5×10⁷, 5.5×10⁷, 6×10⁷, 6.5×10⁷, 7×10⁷, 7.5×10⁷, 8×10⁷, 8.5×10⁷, 9×10⁷, 9.5×10⁷, 1×10⁸, 1.5×10⁸, 2×10⁸, 2.5×10⁸, 3×10⁸, 3.5×10⁸, 4×10⁸, 4.5×10⁸, 5×10⁸, 5.5×10⁸, 6×10⁸, 6.5×10⁸, 7×10⁸, 7.5×10⁸, 8×10⁸, 8.5×10⁸, 9×10⁸, 9.5×10⁸, 1×10⁹, 1.5×10⁹, 2×10⁹, 2.5×10⁹, 3×10⁹, 3.5×10⁹, 4×10⁹, 4.5×10⁹, 5×10⁹, 5.5×10⁹, 6×10⁹, 6.5×10⁹, 7×10⁹, 7.5×10⁹, 8×10⁹, 8.5×10⁹, 9×10⁹, 9.5×10⁹, 1×10¹⁰, 1.5×10¹⁰, 2×10¹⁰, 2.5×10¹⁰, 3×10¹⁰, 3.5×10¹⁰, 4×10¹⁰, 4.5×10¹⁰, 5×10¹⁰, 5.5×10¹⁰, 6×10¹⁰, 6.5×10¹⁰, 7×10¹⁰, 7.5×10¹⁰, 8×10¹⁰, 8.5×10¹⁰, 9×10¹⁰, 9.5×10¹⁰, 1×10¹¹, 1.5×10¹¹, 2×10¹¹, 2.5×10¹¹, 3×10¹¹, 3.5×10¹¹, 4×10¹¹, 4.5×10¹¹, or 5×10¹¹ cells) per m² of body surface of the subject per day.

According to some embodiments of the present disclosure, the subject is a mouse. In certain embodiments, the engineered NK cell is administered to the subject in the amount of 1×10⁶ to 5×10¹¹ cells per m² of body surface of the subject per day; preferably, 1×10⁷ to 5×10¹⁰ cells per m² of body surface of the subject per day; more preferably, 1×10⁸ to 5×10⁹ cells per m² of body surface of the subject per day. The engineered NK cell may be administered to the subject 2-4 times (e.g., 2, 3 or 4 times; preferably, 2 times) per week for 4 weeks, or be administered to the subject 4-6 times (e.g., 4, 5 or 6 times; preferably, 5 times) in the first week, 1-3 times (e.g., 1, 2 or 3 times; preferably, 2 times) in the second week, and 1-3 times (e.g., 1, 2 or 3 times; preferably, 1 time) in the third week. Alternatively, the engineered NK cell may be administered to the subject in the amount of 1×10⁴ to 1×10¹¹ cells per day; preferably, 1×10⁵ to 1×10¹⁰ cells per day; more preferably, 1×10⁶ to 1×10⁹ cells per day, in which the engineered NK cell may be administered to the subject once every 5-8 days (for example, once every 5, 6, 7 or 8 days) for at least 1 month. In one working example, the engineered NK cell is administered to the subject once every 4 days.

In the case when the subject is a human, the engineered NK cell is administered in the amount of 1-5×10⁹ cells per m² of body surface of the subject per day. According to some embodiments, the engineered NK cell is administered to the subject for two consecutive days. According to certain embodiments, the engineered NK cell is administered to the subject in one or more treatment cycles with an interval of about 12 hours to several months between treatments. Depending on desired effects, the interval of treatments may be 12, 13, 14, 15, 16, 17, 18, 19, 20, 21, 22 or 23 hours; be 1, 2, 3, 4, 5, 6, 7, 8, 9, 10, 15, 20 or 25 days; or be 1, 2, 3, 4 or more months. Preferably, the engineered NK cell is administered to the subject on days 1, 3 and 5 of each cycle of treatment. For example, the engineered NK cells may be administered to the subject in the amount of 1×10⁹ cells/m² body surface on day 1 of treatment, 3×10⁹ cells/m² body surface on day 3 of treatment, and 5×10⁹ cells/m² body surface on day 5 of treatment.

Alternatively, the actual dosage of the present agent and engineered NK cell may be determined by the attending physician based on the physical and physiological factors of the subject, these factors include, but are not limited to, age, gender, body weight, body surface, the disease to be treated, severity of the condition, previous history, the presence of other medications, the route of administration and etc.

Non-limiting routes of administration include, but are not limited to, enteral, oral, nasal, parenteral, topical or transmucosal administration, in which the parenteral administration can be any of intratumoral, intramuscular, intravenous, intraarterial, subcutaneous, intraperitoneal, intracerebral, intracerebroventricular or intrathecal injection.

According to embodiments of the present disclosure, the engineered NK cell having the TAA-specific CAR expressed thereon exhibits binding affinity and specificity to the cancer cell treated or pre-treated with the present agent. In these embodiment, the present agent may either additively or synergistically enhance the anti-tumor effect of the present engineered NK cell.

As could be appreciated, the present agent may be administered to the subject before or concurrent with the administration of the engineered NK cell. Preferably, the present agent is administered to the subject before the treatment of the engineered NK cells. Optionally, the present agent is administered in at least 2 independent dosages followed by the treatment of the engineered NK cell. For example, the present agent may be administered to the subject 3, 4, 5, 6, 7, 8, 9, 10 or more times, with each dosage being administered about 1 day apart; and then administered with the engineered NK cell for 1, 2, 3 or more times.

The cancer treatable by the present pharmaceutical kit and/or kit may be resistant to a chemotherapy (e.g., 5-fluorouracil (5-FU)), a radiation therapy (e.g., ultraviolet (UV) radiation) or an immunotherapy (e.g., adoptive immune cell therapy (AIT)). Accordingly, the present pharmaceutical kit and/or method provides a potential means to treat the cancer patient who has developed resistance to cancer therapies.

Alternatively, the present engineered NK cell may be separately administered to the cancer patient without the treatment (e.g., co-treatment or pre-treatment) of the present agent. More specifically, in the case where the TAA expression in a cancer patient is higher than that in a healthy subject, then the cancer patient can be directly treated with the present engineered NK cell without the administration of agent. For example, when the CEA expression level in a cancer patient increases as compared to a healthy subject, then an engineered NK cell comprising the CEA-specific CAR (e.g., the CAR comprises a variable domain, which comprises the amino acid sequence of SEQ ID NO: 1) may be administered to the cancer patient so as to annihilate the CEA-expressing cancer cells.

Exemplary cancers treatable by the present engineered NK cell, pharmaceutical kit and/or kit include, but are not limited to, gastric cancer, lung cancer, bladder cancer, breast cancer, pancreatic cancer, renal cancer, colon cancer, rectal cancer, cervical cancer, ovarian cancer, brain tumor, prostate cancer, hepatocellular carcinoma, melanoma, esophageal carcinoma, multiple myeloma, and head and neck squamous cell carcinoma. According to one specific example of the present disclosure, the cancer is colon cancer or rectal cancer.

Basically, the subject is a mammal, for example, a human, a mouse, a rat, a hamster, a guinea pig, a rabbit, a dog, a cat, a cow, a goat, a sheep, a monkey, and a horse. Preferably, the subject is a human.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### Example

Materials and Methods

Preparing NK92MI-CEA Cells

The NK92MI-CEA cells were produced by following description. The sequences respectively encoding the variable regions of heavy chain (V_{H}) and light chain (V_{L}) of mAb T84.66 were amplified and assembled by overlapping PCR reaction. The sequences encoding the anti-CEA scFv fragment and the hinge region of CD8a (amino acids 105-165) were cloned into plasmid pcDNA3.1/V5-HIS©TOPO®TA. The complete CAR sequence was derived from the resulting pcDNA3.1-scFv (anti-CEA)-CD8a-CD3z construct and cloned into a modified retroviral pLNCX vector, which comprised a leader sequence and an HA tag, via SfiI and Clal cloning sites so as to produce the recombinant retroviral vector pLNCX-scFv (anti-CEA antibody)-CD8α-CD3ζ. The pLNCX-scFv (anti-CEA antibody)-CD8α-CD3ζ was then co-transfected with the pVSV-G plasmid (envelope plasmid) into the packaging cell line GP2-293. The supernatant containing retroviral particles was harvested from the culture medium 48 hours post-transfection, and filtered with a 0.45 μm low-protein binding filter. Next, the filtered supernatant was added to the NK92MI cell line in the presence of polybrene (5 μg/mL). After incubating at 37° C. for 24 hours, the transduced NK92MI cells were screened by neomycin sulfate-G418 (500 mg/ml) so as to produce the NK92MI-CEA cells, which had a CAR (SEQ ID NO: 4) specific to tumor antigen CEA.

Cell Culture

Human colon cancer cell line LS174T (ATCC® CL-188™) and WiDr cells (ATCC CCL-218™) were maintained in alpha modification of Eagle's minimum essential medium (α-MEM) containing 1.5 g/L sodium bicarbonate and 10% fetal bovine serum (FBS). HCT116 cells (ATCC® CCL-247™) were cultured in McCoy's 5A medium containing 1.5 g/L sodium bicarbonate, 4.5 g/L glucose, 10 mM HEPES, 1.0 mM sodium pyruvate and 10% FBS. The NK92MI and NK92MI-CEA cells were cultured in α-MEM supplemented with 1.5 g/L sodium bicarbonate, 0.2 mM inositol, 0.02 mM folic acid, 0.01 mM 2-mercaptoethanol, 10% FBS and 12.5% horse serum. All cells were incubated at 37° C. in a humidified incubator with 5% CO₂.

Determination of CEA Expression

The expression level of CEA on cancer cells were determined by flow cytometry. Cancer cells were stained with the human CEA-specific antibody followed by the analysis of flow cytometry. The fluorescence intensities of at least 10⁵ cells were recorded and analyzed by software. Geometric mean was chosen as mean fluorescence intensity (MFI).

Cytotoxic Assay

In this experiment, the cancer cells (i.e., HCT116 or WiDr cells) and the NK cells (i.e., NK92MI or NK92MI-CEA cells) respectively served as the target and effector cells. In brief, the target cells were co-incubated with the effector cells at various effector/target ratios (E/T), including 10:1, 5:1, 1:1, and 0.5:1, in a round-bottom 96-well culture plate. After incubating at 37° C. for 24 hours, 50 μl of the supernatant was isolated and mixed with 50 μl of CYTOTOX 96® Reagent in a flat-bottom 96-well enzymatic assay plate. The mixture was incubated at room temperature for 30 minutes followed by adding 50 μl stop solution (H₂SO₄) to stop the reaction. The absorbance was measured at 490 nm. The percentage of cytotoxicity for each effector:target cell ratio was calculated by the equation of,

(Experimental−culture medium background)−(Effector cell spontaneous release−culture medium background)−(Target spontaneous release−culture medium background)/(Target maximum release−volume correction control−Target spontaneous release−culture medium background)×100.

Animal Study

2×10⁶ WiDr cells were subcutaneously implanted on the back of 9-weeks-old SCID mice. When tumors reached a volume of 100-200 mm³, the mice were intraperitoneally administered with 200 mg/kg sodium butyrate for 5 consecutive days. Then, the mice were assigned into five groups: (1) control group, in which the mice were orally administrated with PBS every day, and intraperitoneally administrated with PBS every 4 days; (2) NaB group, in which the mice were orally administrated with 5 g/kg sodium butyrate every day, and intraperitoneally administrated with PBS every 4 days; (3) NK92MI group, in which the mice were orally administrated with PBS every day, and intraperitoneally administrated with NK92MI cells every 4 days; (4) NK92MI-CEA group, in which the mice were orally administrated with PBS every day, and intraperitoneally administration with NK92MI-CEA cells every 4 days; and (5) NK92MI-CEA+NaB group, in which the mice were orally administration with 5 g/kg sodium butyrate every day, and intraperitoneally administration with NK92MI-CEA every 4 days. Tumor volume of the mice was measured every 2-3 days, and tumor volumes on were calculated using the formula: length×(width)²÷2 (*p<0.05).

Statistics

For in vivo experiments, tumor volumes were compared using One-Way Anova test with Bonferroni post hoc tests for multiple comparison.

### Example 1 Correlation of CEA Expression and Drug Resistance

It has been reported that the expression of CEA was correlated with the resistance of chemotherapy. The effect of CEA overexpression on 5-fluorouracil resistance was investigated in this example.

HCT116 and WiDr cells were co-treated with sodium butyrate (0.1 mM) or 5-azacytidine (1 μM) and different concentration of 5-fluorouracil (1.2, 2.4, 4.8, 9.6, and 19.2 μM) for 72 hours. Compared to 5-fluorouracil treatment alone, the IC₅₀ value of 5-FU were higher in cells co-treated with sodium butyrate or 5-azacytidine (Tables 1 and 2). The data demonstrated that the treatment of 5-azacytidine or sodium butyrate induced drug resistance in cancer cells.

TABLE 1
Sodium butyrate or 5-azacytidine induced
drug resistance in HCT116 cells.

| | IC50 of 5-fluorouracil | |
|---|---|---|
| | (μM) | |
| Treatment | Mean ± SD | P-value |
| 5-fluorouracil | 4.39 ± 3.10 | |
| sodium butyrate + | 9.40 ± 6.03 | 0.036 |
| 5-fluorouracil | | |
| 5-azacytidine + | 11.76 ± 9.05 | 0.020 |
| 5-fluorouracil | | |

Results were presented as mean ± SD of three independent experiments, each done in triplicate.
P-value (comparing to 5-fluorouracil treatment group).

TABLE 2
Sodium butyrate or 5-azacytidine induced
drug resistance in WiDr cells.

| | IC50 of 5-fluorouracil | |
|---|---|---|
| | (μM) | |
| Treatment | Mean ± SD | P-value |
| 5-fluorouracil | 4.67 ± 0.55 | |
| sodium butyrate + | 9.20 ± 2.74 | <0.001 |
| 5-fluorouracil | | |
| 5-azacytidine + | 10.81 ± 3.34 | <0.001 |
| 5-fluorouracil | | |

Results were presented as mean ± SD of three independent experiments, each done in triplicate.
P-value (comparing to 5-fluorouracil treatment group).

### Example 2 Effect of NK92MI-CEA Cells on Cancer Cells Positively Expressed CEA Thereon

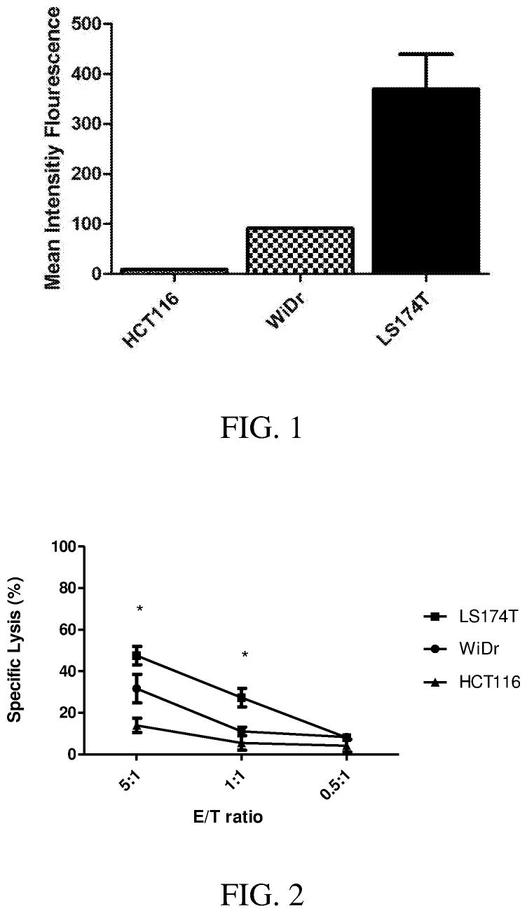

To evaluate whether the expression of CEA would affect the cytotoxic effect of engineered NK cells, HCT cells, WiDr cells and LS174T cells were first subject to flow cytometry to determine the CEA expression thereon. As illustrated in FIG. 1, LS174T had the highest CEA expression as compared to HCT116 and WiDr cells. The cytotoxic effect of NK92MI-CEA cells on the three cancer cell lines was then correlated with their respective CEA expression levels as determined in FIG. 1, and it was found that the percentage of lysed LS174T was significantly higher among the three types of cells (FIG. 2).

The results demonstrated that the NK92MI-CEA cells exhibited binding affinity and cytotoxicity toward CEA-expressing cancer cells, in which the cytotoxic effect was positively correlated with the CEA expression level.

### Example 3 Effect of NK92MI-CEA Cells on Cancer Cells Pretreated with Anti-Cancer Drug

In this example, cancer cells were first treated with various types of anti-cancer drugs, before been subject to the treatment of NK92MI-CEA cells. The data indicated that certain anti-cancer drugs (e.g., 5-azacytidine and sodium butyrate) rendered the cancer cells resistant to the chemotherapy and radiation therapy (data not shown). However, it was also discovered that the cancer therapeutic resistance related to the CEA expression (data not shown). According to the result, administration of 5-azacytidine or sodium butyrate significantly increased CEA expression on cancer cells (data not shown).

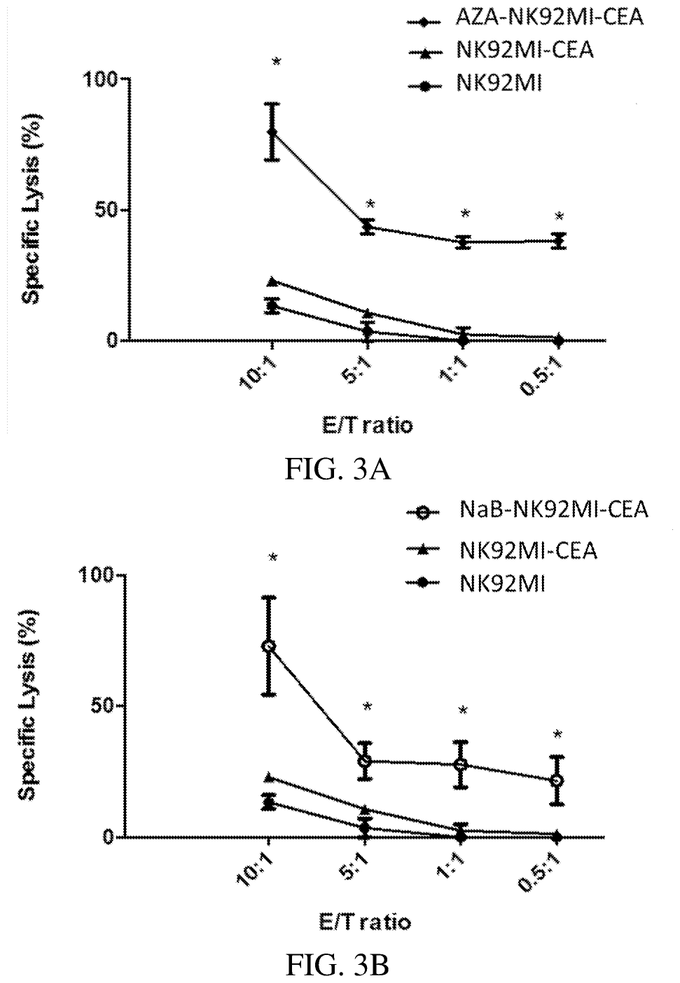

Based on the result, HCT116 cells treated 5-azacytidine or sodium butyrate were co-cultured with NK92MI-CEA cell at an effector/target ratio (E/T ratio) of 10:1, 5:1, 1:1 or 0.5:1. Compared with the control group (i.e., co-incubation of HCT116 and un-engineered NK cell) and untreated group (i.e., co-incubation of HCT116 and engineered NK cell), administration of 5-azacytidine (FIG. 3A) or sodium butyrate (FIG. 3B) significantly enhanced cytotoxic effect of NK92MI-CEA cells.

### Example 4 Effect of NK92MI-CEA Cells in Animal Model

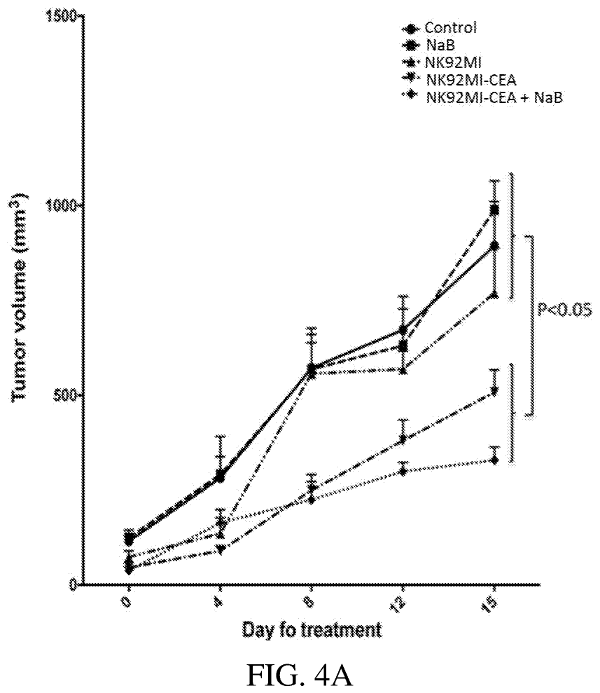

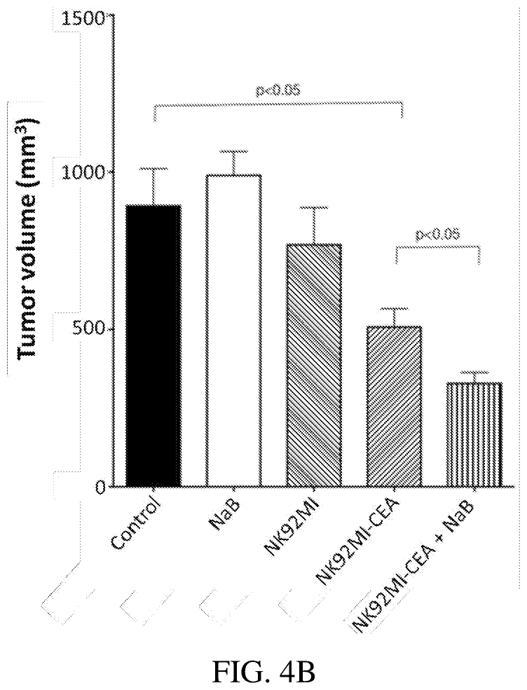

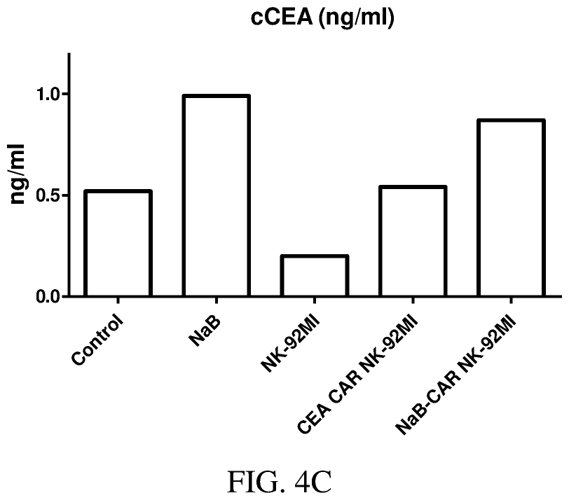

The in vivo anti-tumor activity of NK92MI-CEA cells was evaluated in this example. The mice were treated according to Materials and Methods, and the data was depicted in FIGS. 4A-4C.

The data of FIG. 4A indicated that compared with the control group, the NaB group and the NK92MI group, the treatment of NK92MI-CEA cells significantly inhibit tumor growth. Further, it is noted that the co-treatment of sodium butyrate obviously enhanced the anti-tumor effect of NK92MI-CEA cells.

As the data depicted in FIG. 4B, the treatment of sodium butyrate or NK-92MI cells did not obviously inhibit tumour growth compared to the control group. However, the tumour size was significantly reduced by treatment with NK92MI-CEA cells and even more so in response to the combined treatment of NK92MI-CEA cells and sodium butyrate.

The ELISA data demonstrated that the expression level of serum circulating CEA (cCEA) in mice received sodium butyrate, either sodium butyrate alone or the combination of sodium butyrate and NK92MI-CEA cells, was higher than that of the control mice (i.e., treated with PBS, NK92MI cells or NK92MI-CEA cells) (FIG. 4C). The concentration of cCEA in the serum of mice were respectively 520 pg/ml (control group), 990 pg/ml (NaB group), 200 pg/ml (NK92MI group), 540 pg/ml (NK92MI-CEA group), and 870 pg/ml (NK92MI-CEA+NaB group).

In conclusion, the present disclosure provides a pharmaceutical kit, which comprises a first unit that contains an agent (e.g., 5-azacytidine or sodium butyrate) capable of increasing TAA (e.g., CEA) expression on cancer cells intended to be treated; and a second unit that contains NK cells engineered to express receptors specific for TAA (e.g., NK92MI-CEA cell). The present pharmaceutical kit is useful for treating cancers, especially the cancers that do not respond to conventional cancer treatment; and accordingly, providing a potential means to improve the life quality or lifespan of the cancer patients.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

1. A pharmaceutical kit for treating a cancer in a subject, comprising,
a first container containing an agent that increases the expression of a tumor-associated antigen (TAA) on the cancer; and
a second container containing an engineered natural killer cell having a chimeric antigen receptor (CAR) specific to the TAA.

2. The pharmaceutical kit of claim 1, wherein
the TAA is carcinoembryonic antigen (CEA), and
the CAR comprises a variable domain, which comprises the amino acid sequence at least 85% identical to SEQ ID NO: 1.

3. The pharmaceutical kit of claim 2, wherein the CAR further comprises a hinge domain and an effector domain disposed at the C-terminus of the variable domain, wherein the hinge domain and the effector domain respectively comprises the amino acid sequences at least 85% identical to SEQ ID NOs: 2 and 3.

4. The pharmaceutical kit of claim 1, wherein
the TAA is CEA, and
the CAR comprises the amino acid sequence at least 85% identical to SEQ ID NO: 4.

5. The pharmaceutical kit of claim 1, wherein the agent is selected from the group consisting of, 5-azacytidine, 5,6-dihydro-5-azacytidine, 5-aza-2′-deoxycytidine, arabinofuranosyl-5-azacytosine, trichostatin A, phenylbutyrate, sodium butyrate, valproic acid, and suberoylanilide hydroxamic acid.

6. The pharmaceutical kit of claim 1, wherein the cancer is selected from the group consisting of gastric cancer, lung cancer, bladder cancer, breast cancer, pancreatic cancer, renal cancer, colon cancer, rectal cancer, cervical cancer, ovarian cancer, brain tumor, prostate cancer, hepatocellular carcinoma, melanoma, esophageal carcinoma, multiple myeloma, and head and neck squamous cell carcinoma.

7. The pharmaceutical kit of claim 6, wherein the cancer is resistant to a chemotherapy, radiation therapy or immunotherapy.

8-14. (canceled)

15. A method of treating a cancer in a subject, comprising administering to the subject a first effective amount of an agent that increases the expression of a tumor-associated antigen (TAA) on the cancer, and a second effective amount of an engineered natural killer cell having a chimeric antigen receptor (CAR) specific to the TAA.

16. The method of claim 15, wherein
the TAA is carcinoembryonic antigen (CEA), and
the CAR comprises a variable domain, which comprises the amino acid sequence at least 85% identical to SEQ ID NO: 1.

17. The method of claim 16, wherein the CAR further comprises a hinge domain and an effector domain disposed at the C-terminus of the variable domain, wherein the hinge domain and the effector domain respectively comprises the amino acid sequences at least 85% identical to SEQ ID NOs: 2 and 3.

18. The method of claim 15, wherein
the TAA is CEA, and
the CAR comprises the amino acid sequence at least 85% identical to SEQ ID NO: 4.

19. The method of claim 15, wherein the agent is selected from the group consisting of, 5-azacytidine, 5, 6-dihydro-5-azacytidine, 5-aza-2′-deoxycytidine, arabinofuranosyl-5-azacytosine, trichostatin A, phenylbutyrate, sodium butyrate, valproic acid, and suberoylanilide hydroxamic acid.

20. The method of claim 15, wherein the cancer is selected from the group consisting of gastric cancer, lung cancer, bladder cancer, breast cancer, pancreatic cancer, renal cancer, colon cancer, rectal cancer, cervical cancer, ovarian cancer, brain tumor, prostate cancer, hepatocellular carcinoma, melanoma, esophageal carcinoma, multiple myeloma, and head and neck squamous cell carcinoma.

21. The method of claim 20, wherein the cancer is resistant to a chemotherapy, radiation therapy or immunotherapy.
